(19) United States
(12) Patent Application Publication
(10) Pub. No.: US 2011/0208235 A1
(43) Pub. Date: Aug. 25, 2011

(54) Pedi-Massaggio

(76) Inventor: JOZSEF STERNBACH, Scardale, NY (US)

(21) Appl. No.: 12/709,489

(22) Filed: Feb. 21, 2010

(51) Int. Cl. A61B 17/00

(52) U.S. Cl. 606/204

(57) ABSTRACT

This invention, Pedi-Massaggio , enables anyone to benefit from the stimulating effects of a walk on a pebble beach in the comfort of their home (shower) via the stimulating effects of the sole of the feet.

The Pedi-Massaggio is made of a solid piece of vulcanized rubber. The design is approximately 16″×24″ rectangular shape that features a rock-like top side which stimulates the nerve endings of the feet, as well as provides a non-slip surface when placed in showers or bathtubs.

### BACKGROUND OF INVENTION

This invention, in its creation is indented to enhance the general health of the user as it is documented in the widely available foot reflexology literature. Another important aspect of this invention lies in the safety enhancing quality when used properly in showers and bathtubs.

The designer/inventor of the ‘Pedi-Massaggio’ has no knowledge of any existing product similar to this invention.

### BRIEF SUMMARY OF THE INVENTION

The intended use of this invention (Pedi-Massaggio) is to enhance the general health of the user while providing a non-slip surface in showers and bathtubs.

### BRIEF DESCRIPTION OF DRAWING VIEWS

FIG. 1A—top view

FIG. 1B—cross section view

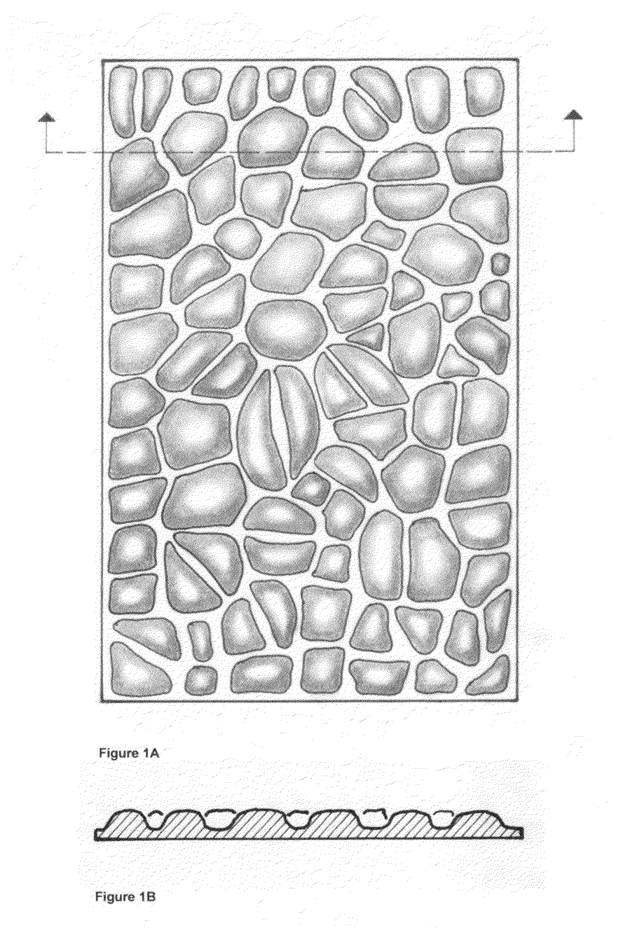

### DETAILED DESCRIPTION OF THE INVENTION

(Description Of The Preferred Embodiment)

Pedi-Massaggio is a unique product that besides the benefits of foot-reflexology it also provides a safe surface when used in showers or bathtubs due to its non-slip surface. This product will be manufactured of solid vulcanized rubber and will be available in many colors.

1. The vulcanized rubber floor mat that is intended to stimulate reflexology points on the bottom of the feet on an artificial rock-like finish, and have the added benefit of a non-slip surface when placed in showers and bathtubs.

2. The invention of this product, as the inventor claims that this product is unique and new; and that no other such similar or like product(s) have been invented.
Further to this claim, the inventor whishes to carry forward with this application by reducing the scope of this claim to include only the features of this invention, should an existing product preclude the inventor's claim of application.
